(19) United States
(12) Patent Application Publication
(10) Pub. No.: US 2003/0069264 A1
(43) Pub. Date: Apr. 10, 2003

(54) METHOD OF DETERMINING AND REDUCING THE RISK OF BPH-RELATED UROLOGIC EVENTS

(76) Inventors: ELIZABETH STONER, WESTFIELD, NJ (US); DANIEL Z. WANG, EDISON, NJ (US); JOANNE WALDSTREICHER, SCOTCH PLAINS, NJ (US)

Correspondence Address: MERCK AND CO INC, P O BOX 2000, RAHWAY, NJ, 070650907

(21) Appl. No.: 09/388,658

(22) Filed: Sep. 2, 1999

Related U.S. Application Data
(60) Provisional application No. 60/099,620, filed on Sep. 9, 1998.

(51) Int. Cl. A61K 31/473; C07D 221/18; G01N 33/53

(52) U.S. Cl. 514/284; 546/77

(57) ABSTRACT

This invention is concerned with a method of determining the risk of a urologic event, particularly an event selected from BPH-related surgery and acute urinary retention in a man by measuring the man's serum PSA level. The present invention also provides for a method of reducing the risk of the urologic event in a man determined to be at risk by the present urologic event risk-determining method by administration to the man of a compound which inhibits 5α-reductase. Also provided is a kit for determining the risk of a urologic event.

### CROSS-REFERENCE TO RELATED APPLICATIONS

The present application claims priority of U.S. provisional application Serial No. 60/099,620, filed Sep. 9, 1998.

### BACKGROUND OF THE INVENTION

Benign prostatic hyperplasia (BPH) is common in older men, with symptoms that impact quality of life, including interference with activities and perception of well being. BPH can be progressive, with risk of BPH-related surgery, urinary retention, infections, bladder calculi and renal failure. Although many men with mild to moderate symptoms do well without intervention, bothersome symptoms and complications can progress in others, leading to medical therapy or surgery.

One of the complications of BPH is acute urinary retention, leading to catheterization. Acute urinary retention (AUR) is a painful condition characterized by the inability to initiate voiding and empty the bladder. Acute urinary retention may be classified as either spontaneous or precipitated. Spontaneous acute urinary retention is often considered by patients to be the most serious outcome of BPH.

Spontaneous acute urinary retention is an episode of acute urinary retention that is due to BPH and is not tied to a precipitating event.

Precipitated acute urinary retention is an episode of acute urinary retention that is precipitated factors other than BPH; for example: anesthesia or surgery within 72 hours; a precipitating medical event such as stroke or congestive heart failure; a medical condition such as prostatitis or urinary tract infection; or ingestion of medication or drugs known to precipitate retention, e.g., pseudoephedrine hydrochloride, cold medicine, pain medication such as narcotics or sedatives, or benadryl.

Surgery for BPH includes balloon dilatation, microwave hyperthermia, laser pro statectomy, open prostatectomy, suprapubic prostatectomy, transurethral incision of the prostate (TUIP), transurethral laser incision of the prostate (TULIP), transurethral microwave thermotherapy (TUMT), transurethral resection of the prostate (TURP), and video laser ablation of the prostate (VLAP). These procedures range from minimally invasive surgery, such as microwave hyperthermia, to the extremely invasive open prostatectomy. Each of these procedures has associated risks and benefits.

Until recently, there was no test to determine who is at high risk for the development of acute urinary retention or surgery for BPH. Physicians relied on their clinical judgment. Recently, Jacobsen (J. Urology, vol. 158, pp. 481-487 (1997)) demonstrated that patients with increased prostate volume had a higher risk of acute urinary retention. Jacobsen did not address the risk of BPH-related surgery.

The present invention is directed to a method for determining the risk for the development of urologic events, such as BPH-related surgery and acute urinary retention, by measuring serum prostate-specific antigen (PSA) levels. The present invention is also directed to a method of reducing the risk for the development of urologic events, such as BPH-related surgery and acute urinary retention, by administering an inhibitor of 5-alpha-reductase to a man determined to be at risk for the development of urologic events via the serum PSA risk reduction method.

Serum PSA is currently the most widely used marker for prostate cancer detection, and a yearly measurement is recommended in men over 50 years old to aid in the early detection of prostate cancer.

The enzyme 5α-reductase catalyzes the reduction of testosterone (T) to the more potent androgen, 5α-dihydrotestosterone (“dihydrotestosterone” or DHT), as shown below:

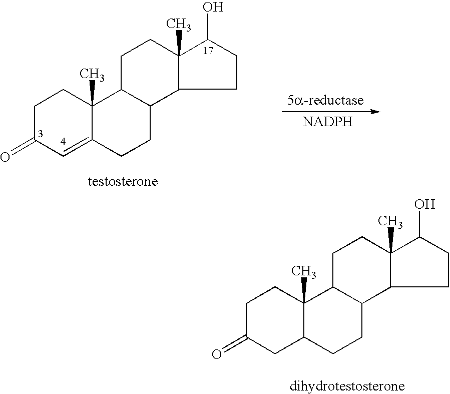

There are two isozymes of 5α-reductase in humans. One isozyme (type 1) predominates in the sebaceous glands of skin tissue. The other (type 2) predominates in the prostate.

Finasteride (17β-(N-tert-butylcarbamoyl)-3-oxo-4-aza-5α-androst-1-en-3-one), as shown below, is a potent inhibitor of the human type 2 enzyme.

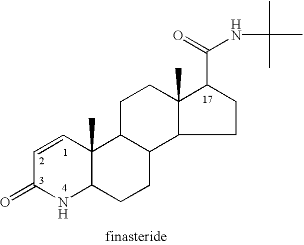

Under the tradename PROSCAR®, finasteride is known to be useful in the treatment of hyperandrogenic conditions, see e.g., U.S. Pat. No. 4,760,071. Finasteride is currently prescribed for the treatment of benign prostatic hyperplasia (BPH), a condition affecting to some degree the majority of men over age 55. Under the tradename PROPECIA®, finasteride is also prescribed for the treatment of male pattern hair loss.

Also known are compounds which are potent inhibitors of both 5α-reductase type 1 and type 2. These include the compound described in U.S. Pat. No. 5,565,467.

### SUMMARY OF THE INVENTION

This invention is concerned with a method of determining the risk of a urologic event, particularly an event selected from BPH-related surgery and acute urinary retention, in a man by measuring the man's serum PSA level. The present invention also provides for a method of reducing the risk of the urologic event in a man determined to be at risk by the present urologic event risk-determining method by administration to the man of a compound which inhibits 5α-reductase.

### DESCRIPTION OF THE DRAWINGS

FIG. 1 is a graph of the cumulative incidences for spontaneous acute urinary retention (AUR) over four years by increments of baseline serum PSA thresholds in the clinical study detailed in Example 1. This graph shows that in the placebo group, the cumulative incidence of spontaneous AUR increases with increasing serum PSA values; and in the finasteride-treated patients, this effect is nearly absent.

FIG. 2 is a graph of the cumulative incidences for all AUR (spontaneous and precipitated combined) over four years by increments of baseline serum PSA thresholds in the clinical study detailed in Example 1. This graph shows that in the placebo group, the cumulative incidence of all AUR increases with increasing serum PSA values; and in the finasteride-treated patients, this effect is nearly absent.

FIG. 3 is a graph of the cumulative incidences BPH-related surgery over four years by increments of baseline serum PSA thresholds in the clinical study detailed in Example 1. This graph shows that in the placebo group, the cumulative incidence of BPH-related surgery increases linearly across PSA values from 10-24%, while it increases only in finasteride-treated patients in men with a baseline PSA above 5.0 ng/mL.

### DETAILED DESCRIPTION OF THE INVENTION

In one embodiment of the present invention is a method of determining the risk of a urologic event in a man by measuring the serum PSA level of the man.

In another embodiment of the present invention is provided a method for determining the risk of a urologic event selected from BPH-related surgery and acute urinary retention in a man by measuring the man's serum PSA level.

In one class of this embodiment is the method for determining the risk of BPH-related surgery in a man by measuring the man's serum PSA level.

In another class of this embodiment is the method for determining the risk of acute urinary retention in a man by measuring the man's serum PSA level.

Another embodiment of the present invention is a method of determining the risk of a urologic event in a man by measuring the serum PSA level of the man and determining if the value is over 3.3 ng/mL.

Another embodiment of the present invention is a method of determining the risk of a urologic event in a man by measuring the serum PSA level of the man and determining if the value is over 1.3 ng/mL.

An additional embodiment of the present invention is a method of reducing the risk of a urologic event in a man determined to have a risk of a urologic event by the present method of determining the risk of the urologic event by the administration to the man of a compound which inhibits 5α-reductase.

In yet another embodiment of the present invention is a method of reducing the risk of a urologic event in a man determined to be at high risk by the present method of determining the risk of a urologic event by the administration to the man of a compound which inhibits 5α-reductase.

Still another embodiment of the present invention is a method of reducing the risk of a urologic event in a man at risk for a urologic event by having a serum PSA level of over 1.3 ng/mL by the administration to the man of a compound which inhibits 5α-reductase.

Yet another embodiment of the present invention is a method of reducing the risk of a urologic event in a man at risk for a urologic event by having a serum PSA level of over 3.3 ng/mL by the administration to the man of a compound which inhibits 5α-reductase.

Still another embodiment of the present invention is a kit for determining the risk of a urologic event in a man comprising a test for serum PSA and for relating the serum PSA measurement to the risk of a urologic event.

In one class of this embodiment, the kit comprises a test for serum PSA and a graph of PSA versus risk of a urologic event.

In another class of this embodiment, the kit comprises a test for serum PSA and a chart relating serum PSA and risk of a urologic event.

In one class of this embodiment, the kit comprises a test for total serum PSA and a graph of total PSA versus risk of a urologic event.

In still another class of this embodiment, the kit comprises a test for total serum PSA or percent free PSA and a chart relating total serum PSA or percent free PSA and risk of a urologic event.

In another class of this embodiment, the kit comprises a test for free serum PSA and a graph of free PSA versus risk of a urologic event.

In yet another class of this embodiment, the kit comprises a test for free serum PSA and a chart relating free PSA versus risk of a urologic event.

In still another class of this embodiment, the kit comprises a test for percent free PSA and a graph of percent PSA versus risk of a urologic event.

Another class of this embodiment, the kit comprises a test for percent free PSA and a chart relating percent PSA versus risk of a urologic event.

Inhibitors of 5α-reductase type 2 are known in the art. For a given compound, its 5α-reductase type 2 inhibitory activity may be determined by assaying its activity as described in Example 3 in the present application. Compounds having an IC₅₀ under about 100 nM are 5α-reductase type 2 inhibitors useful in the present invention. Compounds also having both 5α-reductase type 2 and 5α-reductase type 1 activity, often called “dual inhibitors” are also compounds useful in the methods of the present invention. Further, inhibitors of 5α-reductase type 1 may be useful in the methods of the present invention.

Among the compounds useful in the methods of reducing the risk of urologic events of the present invention are those of structural formula I:

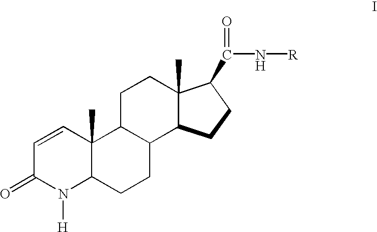

wherein R is selected from:

(a) C₁₋₁₀ alkyl, unsubstituted or substituted with one to three halogen substituents, and

(b) phenyl, unsubstituted or substituted with one to three substituents independently selected from halogen, methyl, and trifluoromethyl.

In one embodiment of compounds of structural formula I, R is selected from

(a) unsubstituted C₁₋₁₀ alkyl, and

(b) phenyl unsubstituted or substituted with one or two trifluoromethyl substituents.

In another embodiment of compounds of structural formula I, R is t-butyl.

In yet another embodiment of compounds of structural formula I, R is 2,5-bis(trifluoromethyl)phenyl.

Other inhibitors of 5α-reductase type 2 useful in the methods of the present invention include epristeride and turosteride, shown below:

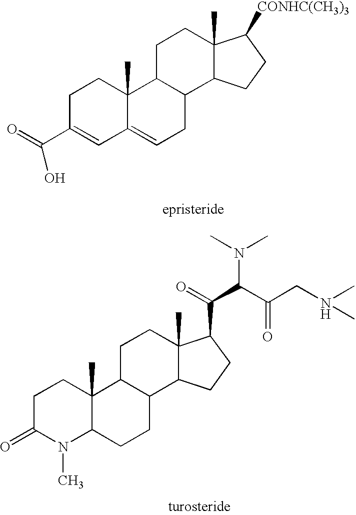

The term “halo” or “halogen” is meant to include fluoro, chloro, bromo and iodo.

The term “C₁₋₁₀ alkyl” is meant to include both straight- and branched-chain alkyl groups of one to ten carbon atoms in length, not limited to: methyl, ethyl, propyl, butyl, pentyl, hexyl, heptyl, octyl, nonanyl, decyl and the isomers thereof such as isopropyl, isobutyl, secbutyl, t-butyl, isopentane, isohexane, etc.

Many organic compounds can form complexes with solvents in which they are reacted or from which they are precipitated or crystallized. These complexes are known as “solvates”. Solvates of compounds of structural formula I are within the scope of the present invention. Many organic compounds can exist in more than one crystalline form. For example, crystalline form may vary from solvate to solvate. Thus, all crystalline forms of the compounds of structural formula I or the pharmaceutically acceptable solvates thereof are within the scope of the present invention.

The method of determining the risk may be employed in an adult male human. The method of reducing the risk may be employed in an adult male human determined by the risk determination method to be at risk for a urologic event.

For example, the method of determining the risk may be employed in a physician's office. An individual male subject having a risk of bleeding may present to the physician. This individual would be at a high risk for complications during surgery. By employing the method of the present invention, the physician could measure the serum PSA of the subject, and determine the risk of the subject for a urologic event. Using this information, the physician is better able to evaluate the subject's risk of having a urologic event requiring surgery in the future and may prescribe a 5α-reductase inhibitor to reduce the risk of surgery if such a risk is high.

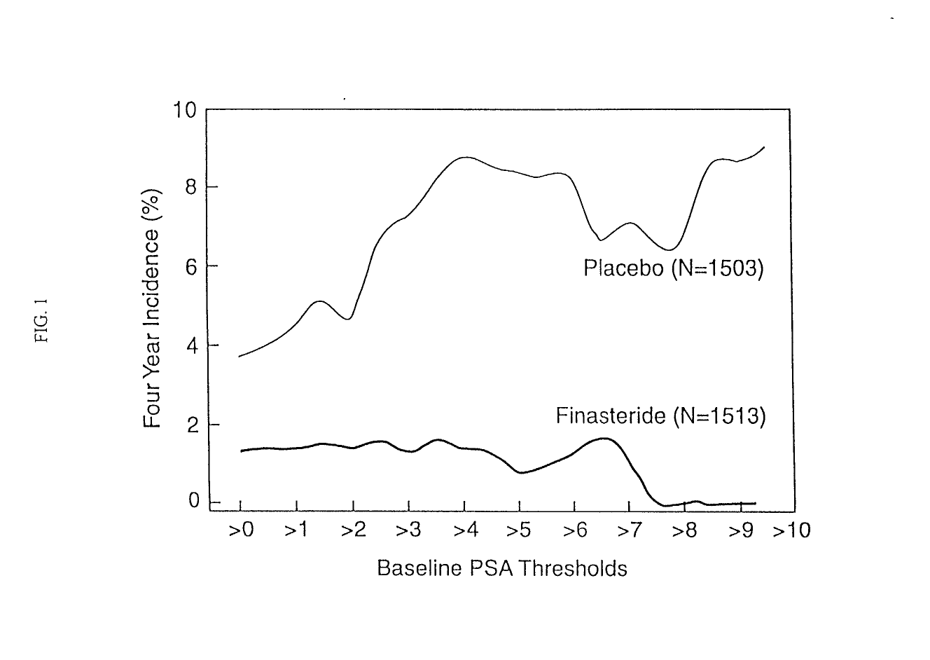

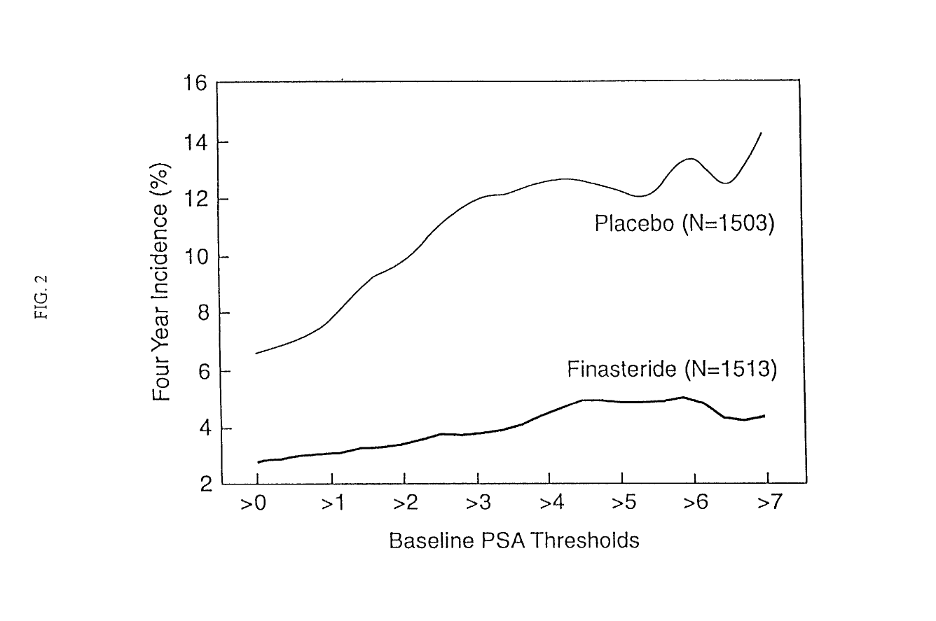

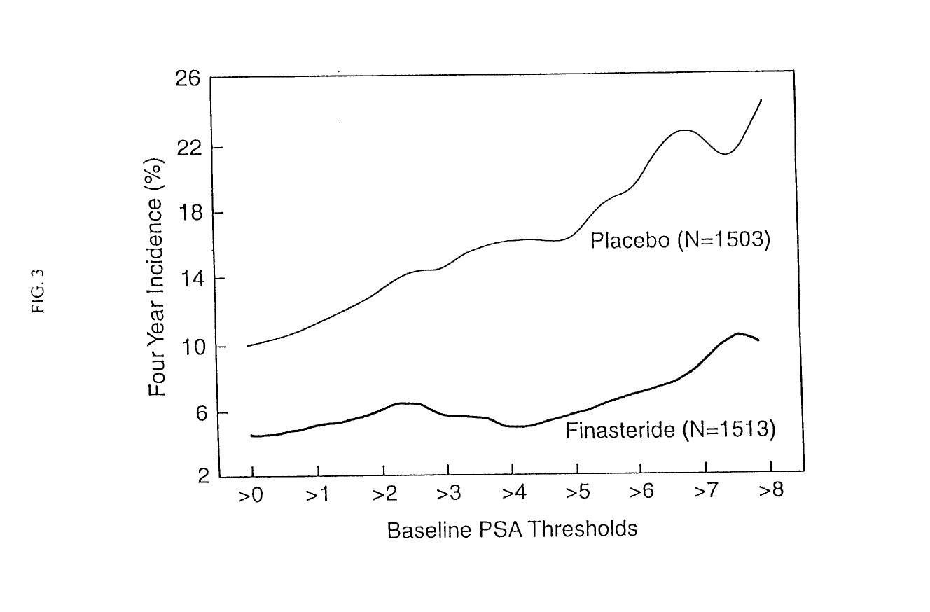

Physicians may have different thresholds regarding their use of preventive health care measures. FIGS. 1, 2, and 3 provide a graphic assessment to aid in this decision. The cumulative incidence of spontaneous AUR for placebo-treated patients increases dramatically above serum PSA levels of approximately 1.3 ng/mL. In fact, while the cumulative risk for all patients is approximately 4% or 1 in 25 men over four years, it reaches 9% or nearly 1 in 10 patients for those with a PSA over 4.0 ng/mL at baseline (FIG. 1). At the same time, the risk remains unchanged over the entire serum PSA spectrum for finasteride-treated patients. Similar observations hold true for the cumulative risk for both spontaneous and precipitated AUR, as well as for BPH-related surgery (FIGS. 2 and 3).

The 5α-reductase inhibitor finasteride attenuates the predictive power of prostate volume and serum PSA regarding surgery and AUR. In fact, to make predictions about the risk of surgery and/or AUR once treatment is initiated is less important than to be able to give patients and health care providers information prior to a treatment decision regarding possible future BPH-related outcomes.

In this context, serum PSA is a good candidate parameter to aid in the individualized discussion that takes place between patients and physicians before initiation of therapy for BPH. Finasteride decreases the risk of developing a BPH-related outcome by approximately half in all subgroups examined. However, the absolute risk of having an outcome is substantially different across the different levels of PSA and prostate volume. Risk is viewed differently by patients, physicians and administrators. The data provided in this report should be helpful to all parties involved in the decision whether or not to treat BPH based on predictable risks and predictable reductions in risk with finasteride.

Serum PSA may be measured by measuring the total amount of PSA per volume of serum by a variety of known methods. In addition, the methods of the present invention may be employed by measuring free PSA per volume of serum or percent free PSA by methods known in the art. Especially preferred are the PSA measurement techniques available from Hybritech.

The term “effective amount” means the amount of 5α-reductase inhibitor that will elicit the biological or medical response of a tissue, system, animal or human that is being sought by the researcher, veterinarian, medical doctor or other clinician. In particular, an “effective amount” for risk reduction means the amount of the 5α-reductase inhibitor that reduces serum PSA (either total PSA, free PSA or percent free PSA) by approximately 50%.

The term “composition” as used herein is intended to encompass a product comprising the specified ingredients in the specified amounts, as well as any product which results, directly or indirectly, from combination of the specified ingredients in the specified amounts.

The inhibitors of 5α-reductase employed in the present invention are useful as pharmacological agents for mammals, especially for humans, for the prevention of and reduction of the risk of precipitated acute urinary retention.

Generally, the daily dosage of the 5α-reductase inhibitor may be varied over a wide range from 0.01 to 500 mg per adult human per day. In a preferred embodiment, the 5α-reductase inhibitor is administered at a dose of 1.0 to 100 mg per day. In another preferred embodiment, the 5α-reductase inhibitor is administered at a dose of 0.5 to 10 mg per day. For oral administration, the compositions are preferably provided in the form of tablets containing 0.01, 0.05, 0.1, 0.5, 1.0, 2.5, 5.0, 10.0, 15.0, 25.0, 50.0 and 100 milligrams of active ingredient for the symptomatic adjustment of the dosage to the subject to be treated. An effective amount of the drug is ordinarily supplied at a dosage level of from about 0.0002 mg/kg to about 50 mg/kg of body weight per day. The range is more particularly from about 0.001 to 7 mg/kg of body weight per day.

The dose may be administered in a single daily dose or the total daily dosage may be administered in divided doses of two, three or four times daily. Furthermore, when administered via intranasal routes, transdermal routes, by rectal suppositories, or through a continual intravenous solution, the dosage administration will, of course, be continuous rather than intermittent throughout the dosage regimen.

In the methods of the present invention, the inhibitor of 5α-reductase inhibitor may preferably be administered to the individual to avoid the risk for precipitated acute urinary retention and to reduce the risk of having such an episode in the future. Alternatively, the administration of the 5α-reductase inhibitor may be commenced before a scheduled precipitating event (such as surgery or anesthesia) to prevent the occurrence of acute urinary retention related to the event.

Formulations of the 5α-reductase inhibitors employed in the present method for medical use comprise the 5α-reductase inhibitor together with an acceptable carrier thereof. The carrier must be pharmaceutically acceptable in the sense of being compatible with the other ingredients of the formulation and not deleterious to the recipient subject of the formulation.

According to the methods of the present invention, the 5α-reductase inhibitor may be administered as the sole active agent or together with another active agent such as an antiandrogen, a GnRH analog, a GnRH antagonist or with another 5α-reductase inhibitor.

The present invention, therefore, further provides a pharmaceutical formulation comprising a 5α-reductase type 2 inhibitor together with a pharmaceutically acceptable carrier thereof.

The formulations include those suitable for oral, rectal, topical or parenteral (including subcutaneous, intramuscular and intravenous administration). Preferred are those suitable for oral administration.

The formulations may be presented in a unit dosage form and may be prepared by any of the methods known in the art of pharmacy. All methods include the step of bringing the active compound in association with a carrier which constitutes one or more ingredients. In general, the formulations are prepared by uniformly and intimately bringing the active compound in association with a liquid carrier, a waxy solid carrier or a finely divided solid carrier, and then, if needed, shaping the product into the desired dosage form.

According to the formulations of the present invention, the 5α-reductase inhibitor may be the sole active agent or may be present together with another active agent such as an antiandrogen, a GnRH analog, a GnRH antagonist or with another 5α-reductase inhibitor. Alternatively, treatment may encompass administration of a combination of a compound of formula I with a 5α-reductase 2 inhibitor and/or another active agent such as an alpha1 or an alpha1a adrenergic receptor antagonist (alpha1a receptor antagonists were formerly called alpha1c receptor antagonists). In another embodiment, the 5α-reductase inhibitor may be administered together with an endothelin antagonist.

Formulations of the present invention suitable for oral administration may be presented as discrete units such as capsules, cachets, tablets or lozenges, each containing a predetermined amount of the active compound; as a powder or granules; or a suspension or solution in an aqueous liquid or non-aqueous liquid, e.g., a syrup, an elixir, or an emulsion.

A tablet may be made by compression or molding, optionally with one or more accessory ingredients. Compressed tablets may be prepared by compressing in a suitable machine the active compound in a free flowing form, e.g., a powder or granules, optionally mixed with accessory ingredients, e.g., binders, lubricants, inert diluents, disintegrating agents or coloring agents. Molded tablets may be made by molding in a suitable machine a mixture of the active compound, preferably in powdered form, with a suitable carrier.

Suitable binders include, without limitation, starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include, without limitation, sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

Oral liquid forms, such as syrups or suspensions in suitably flavored suspending or dispersing agents such as synthetic and natural gums, for example, tragacanth, acacia, methyl cellulose and the like may be made by adding the active compound to the solution or suspension. Additional dispersing agents that may be employed include glycerin and the like.

Formulations for rectal administration may be presented as a suppository with a conventional carrier, i.e., a base that is nontoxic and nonirritating to mucous membranes, compatible with the 5α-reductase inhibitors, and is stable in storage and does not bind or interfere with the release of the compound. Suitable bases include: cocoa butter (theobroma oil), polyethylene glycols (such as carbowax and polyglycols), glycol-surfactant combinations, polyoxyl 40 stearate, polyoxyethylene sorbitan fatty acid esters (such as Tween, Myrj, and Arlacel), glycerinated gelatin, and hydrogenated vegetable oils. When glycerinated gelatin suppositories are used, a preservative such as methylparaben or propylparaben may be employed.

Topical preparations containing the active drug component can be admixed with a variety of carrier materials well known in the art, such as, e.g., alcohols, aloe vera gel, allantoin, glycerine, vitamin A and E oils, mineral oil, PPG2 myristyl propionate, and the like, to form, e.g., alcoholic solutions, topical cleansers, cleansing creams, skin gels, skin lotions, and shampoos in cream or gel formulations.

The compounds of the present invention can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Compounds of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamidephenol, polyhydroxy-ethylaspartamidephenol, or polyethylene-oxide polylysine substituted with palmitoyl residues. Furthermore, the compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydropyrans, polycyanoacrylates and cross-linked or amphipathic block copolymers of hydrogels.

Formulations suitable for parenteral administration include formulations that comprise a sterile aqueous preparation of the active compound that is preferably isotonic with the blood of the recipient. Such formulations suitably comprise a solution or suspension of a compound that is isotonic with the blood of the recipient subject. Such formulations may contain distilled water, 5% dextrose in distilled water or saline and the active compound. Often it is useful to employ a pharmaceutically and pharmacologically acceptable acid addition salt of the active compound that has appropriate solubility for the solvents employed. Useful salts include the hydrochloride isothionate and methanesulfonate salts. Useful formulations also comprise concentrated solutions or solids comprising the active compound which on dilution with an appropriate solvent give a solution suitable for parenteral administration.

The compounds of the present invention may be coupled to a class of biodegradable polymers useful in achieving controlled release of a drug, for example, polylactic acid, polyepsilon caprolactone, polyhydroxy butyric acid, polyorthoesters, polyacetals, polydihydro-pyrans, polycyanoacrylates and cross-linked or amphipathic block copolymers of hydrogels.

Alpha-1 adrenergic receptor antagonists suitable for administration together with the 5α-reductase inhibitors according to the method fo the present invention include those such as e.g., terazosin, doxazosin, prazosin, bunazosin, indoramin or alfuzosin, may be employed. More particularly, the combined therapy can comprise administering a 5α-reductase, such as e.g., finasteride, and an alpha-1a adrenergic receptor antagonist (formerly called an alpha-1c adrenergic receptor antagonist). Compounds which are useful as alpha-1a adrenergic receptor antagonists can be identified according to procedures known to those of ordinary skill in the art, for example, as described in U.S. Pat. No. 5,403,847.

The tertile analysis presented in the present invention demonstrates a very strong relationship between serum PSA and the prediction of the incidence of BPH-related surgery or AUR over 4 years. The significant increase in the risk of experiencing these complications with placebo treatment is nearly completely obliterated with finasteride treatment. This phenomenon leads to a greater benefit of finasteride over placebo in men with either larger prostate volumes or higher baseline PSA in avoiding these complications. About 1 out of 5 patients in the highest tertiles are likely to experience either one of the two complications, and the risk reduction with finasteride is 60% based on serum PSA.

In placebo-treated patients, Example 1 demonstrates that the cumulative risk increases in a linear fashion for all three serum PSA tertiles. With the exception of the lowest PSA tertile where there is no apparent benefit of finasteride over placebo in the first two years, the difference in risk between treatment groups in the higher tertiles (>1.3 ng/mL) becomes evident as early as the first follow-up visit at 4 months.

FIGS. 1, 2, and 3 provide a graphic assessment to aid in the decision of setting a threshold for preventive health care measures. The cumulative incidence of spontaneous AUR for placebo-treated patients increases dramatically above of a cut-point of serum PSA of approximately 1.3 ng/mL. In fact, while the cumulative risk for all patents is approximately 4% or 1 in 25 men over 4 years, it reaches 9% or nearly 1 in 10 patients for those with a PSA over 4.0 ng/mL at baseline. At the same time, the risk remains unchanged over the entire serum PSA spectrum for finasteride-treated patients. Similar observations hold true for the cumulative risk for both spontaneous and precipitated AUR as well as for BPH-related surgery.

The following examples are not intended to be limitations on the scope of the instant invention in any way, and they should not be so construed. Furthermore, examples are not to be construed as forming the only methods and compositions that are considered as the invention. Those skilled in the art will readily understand that known variations of the conditions, processes, methods and compositions of the following preparative procedures can be used.

### EXAMPLE 1

Effect of the 5α-reductase Inhibitor Finasteride on the Risk of Precipitated Acute Urinary Retention

A total of 3040 men with clinical BPH diagnosed on the basis of moderate-to-severe symptoms, a decreased peak urinary flow rate (less than 15 mL/sec with a voided volume of 150 mL or more) and an enlarged prostate gland by digital rectal examination (DRE) were enrolled in a four-year study comparing finasteride with placebo. Men receiving alpha blocking agents or antiandrogens, and men with a history of chronic prostatitis, recurrent urinary tract infections, prostate or bladder cancer or surgery, or a serum PSA over 10 ng/mL were excluded. Men with serum PSA concentrations between 4.0 and 9.9 ng/mL had to have a negative prostate biopsy prior to enrollment.

After a one-month single-blind placebo lead-in, men were randomly assigned to receive placebo or 5 mg of finasteride (17β-(N-tert-butylcarbamoyl)-3-oxo-4-aza-5α-androst-1-en-3-one) daily in a four year, double-blind placebo-controlled study. BPH-related outcomes including symptoms, bothersomeness, adverse events and urinary flow rates, were assessed every 4 months. Serum PSA was measured every 4 months in the first year and then every 8 months at a central laboratory. Physical examination and routine hematological and serum chemistry tests were performed yearly. Magnetic resonance imaging (MRI) was performed at baseline and subsequently yearly in a subset of 10% of patients. All MRI images were read by a central radiologist blinded to treatment allocation and time of imaging.

Acute urinary retention (AUR) and surgery for BPH were predefined secondary end-points. The endpoint committee, blinded to treatment group, reviewed all study-related documents related to episodes of acute urinary retention, and all prostate surgeries for BPH, excluding surgery for prostate cancer. The endpoint committee classified episodes of AUR as spontaneous versus precipitated (when contributing factors such as urinary tract infection, surgery anesthesia, ingestion of alpha sympathomimetic drugs or anticholinergics were identified).

Complete data on outcomes, including four-year follow-up information for men who had discontinued treatment, were available for 92% of the men randomized. In the other 8%, complete information was available until discontinuation of the medication or up to the 6-month follow-up assessment after discontinuation.

The effect of baseline serum PSA on the risk of BPH-related outcomes (developing AUR and need for BPH-related surgery), were assessed by dividing patients into tertiles of baseline serum PSA, and calculating the risk of developing an outcome by time-to-first event analysis as well as Fisher's exact test for cumulative incidence. The finasteride-related reduction in risk over 4 years was calculated using log rank analyses. All statistical tests were two-sided and a p-value of <0.05 accepted as significant.

The measurement characteristics of baseline in prediction of BPH-related outcomes were evaluated using receiver operating characteristic (ROC) curves. The area under the ROC curve (AUC) for a given population is given by the probability that a randomly chosen individual in the affected population has a higher value of the index (in this case PSA) than a randomly chosen individual in the non-affected population. The AUC's were computed using the method of Hanley and McNeil. Differences between AUC's in the finasteride and placebo group were tested using normal statistics with standard deviations computed by the same method.

At baseline, men assigned to finasteride and placebo were similar in terms of age, demographics, symptom severity, peak flow rate, prostate volume and serum PSA. Baseline characteristics of the subset with prostate volume measurements were similar to those in the entire study group.

The overall incidence of acute urinary retention was 7% on placebo and 4% on finasteride (spontaneous AUR 4% on placebo and 1% on finasteride; precipitated AUR 3% on placebo and 2% on finasteride) and of BPH-related surgery 10% in men on placebo and 5% in men on finasteride.

When stratified by baseline serum PSA, the risk increased from 7.8% to 19.9% for the placebo group (p<0.001) and from 4.4% to 8.3% for the finasteride group (p=0.035), resulting in a finasteride-related reduction of risk from 43% in the lowest to 60% in the highest tertile of baseline serum PSA.

Patients in the lowest tertile of serum PSA (0.0-1.3 ng/mL) had the lowest risk for either AUR or surgery, and the benefit of finasteride over placebo was minimal for the first two years. In this tertile, the overall 4-year relative risk for finasteride versus placebo-treated patients is 0.57 (95% CI 0.35 to 0.95) with a 43% risk reduction (p=0.030)

For patients in the second tertile (serum PSA between 1.4 and 3.2 ng/mL), the 4 year risk for finasteride versus placebo-treated patients is 0.54 (95% CI 0.37 to 0.80), with a 46% risk reduction with finasteride treatment (p=0.002).

Patients in the third tertile of serum PSA(3.3-12 ng/mL) had a 4-year relative risk for finasteride versus placebo treated patients of 0.40 (95% CI 0.29 to 0.56), with a 60% risk reduction on finasteride (p<0.001). About 1 out of 5 patients in the highest tertile is likely to experience either urinary retention or surgery for BPH, and the risk reduction with finasteride treatment is 60% (based on serum PSA).

FIGS. 1, 2, and 3 show the cumulative incidences for spontaneous AUR (FIG. 1), all AUR (spontaneous and precipitated combined) (FIG. 2), and BPH-related surgery (FIG. 3) over four years by increments of baseline serum PSA thresholds.

In the placebo group the cumulative incidence for spontaneous (FIG. 1) and all AUR (FIG. 2) increases with increasing serum PSA values (p<0.001); whereas in the finasteride-treated group, this effect is nearly absent. The cumulative incidence of BPH related surgery increases linearly across PSA values in placebo treated patients from 10 to 24% (p<0.001), while it increases only in finasteride-treated patients in men with a baseline PSA above 5.0 ng/mL (p=NS) (FIG. 3). These data strongly suggest that the increased risk for AUR and/or surgery with increasing baseline serum PSA is nearly obliterated with finasteride therapy.

ROC curve analyses evaluating the performance of baseline serum PSA in predicting outcomes in comparison to the more traditional baseline parameters of BPH, including symptom severity, bothersomeness, peak urinary flow rate, residual urine volume and age are shown in Table 1, below. Table 1 AUC (area under the curve)±standard error values for ROC curves for several baseline parameters for spontaneous AUR (acute urinary retention) and prostate related surgery

| | | | |
|---|---|---|---|
| Parameter | Placebo | Finasteride | P-value [symbol] |

Spontaneous Acute Urinary Retention

| | | | |
|---|---|---|---|
| Serum PSA | 0.70 ± 0.03 | 0.53 ± 0.06 | 0.012 |
| Prostate volume | 0.81 ± 0.12 | 0.67 ± 0.30 | 0.665 |
| Peak flow rate | 0.62 ± 0.04 | 0.67 ± 0.06 | 0.510 |
| Residual urine volume | 0.56 ± 0.04 | 0.46 ± 0.07 | 0.224 |
| Quasi AUA symptom score | 0.55 ± 0.04 | 0.49 ± 0.06 | 0.440 |
| Bothersomeness score | 0.58 ± 0.04 | 0.46 ± 0.07 | 0.168 |
| Age | 0.53 ± 0.04 | 0.57 ± 0.06 | 0.562 |

BPH-Related Surgery

| | | | |
|---|---|---|---|
| Serum PSA | 0.62 ± 0.02 | 0.59 ± 0.03 | 0.461 |
| Prostate volume | 0.63 ± 0.08 | 0.49 ± 0.09 | 0.213 |
| Peak flow rate | 0.57 ± 0.03 | 0.59 ± 0.04 | 0.692 |
| Residual urine volume | 0.60 ± 0.02 | 0.52 ± 0.03 | 0.046 |
| Quasi AUA symptom score | 0.59 ± 0.02 | 0.60 ± 0.03 | 0.761 |
| Bothersomeness score | 0.61 ± 0.02 | 0.55 ± 0.03 | 0.197 |
| Age | 0.57 ± 0.03 | 0.60 ± 0.03 | 0.507 |

Á Represents the between group P-value

Within the placebo group, serum PSA and prostate volume were the best predictors of all AUR (AUCs 0.68 and 0.69, respectively), as well as for spontaneous AUR (AUC 0.70 and 0.81, respectively). Interestingly, serum PSA, prostate volume, and peak flow rate had higher AUC values in the placebo group for AUR than for BPH-related surgery. Symptom severity, bothersomeness scores, and residual urine had higher AUC values in the placebo group for BPH-related surgery than for AUR. Age had similar AUC values in the placebo group for both AUR and BPH-related surgery. In general the AUCs were numerically higher for the placebo group than for the finasteride group, suggesting that finasteride treatment weakens the relationship between baseline values and the occurrence of BPH-related outcomes. Interestingly, neither symptom severity nor bothersomeness of symptoms had any predictive value (AUCs 0.46 and 0.48, respectively) for the development of AUR in finasteride-treated patients (Table 1).

### EXAMPLE 2

Preparation of Human Prostatic and Scalp 5α-Reductases

Samples of human tissue were pulverized using a freezer mill and homogenized in 40 mM potassium phosphate, pH 6.5, 5 mM magnesium sulfate, 25 mM potassium chloride, 1 mM phenylmethyl-sulfonyl fluoride, 1 mM dithiothreitol (DTT) containing 0.25 M sucrose using a Potter-Elvehjem homogenizer. A crude nuclear pellet was prepared by centrifugation of the homogenate at 1,500×g for 15 min. The crude nuclear pellet was washed two times and resuspended in two volumes of buffer. Glycerol was added to the resuspended pellet to a final concentration of 20%. The enzyme suspension was frozen in aliquots at −80° C. The prostatic and scalp reductases were stable for at least 4 months when stored under these conditions.

### EXAMPLE 3

5α-Reductase Assay

The reaction mixture for the type 1 5α-reductase contained 40 mM potassium phosphate, pH 6.5, 5 mM [7-³H]-testosterone, 1 mM dithiothreitol and 500 μM NADPH in a final volume of 100 μL. The reaction mixture for the type 2 5α-reductase contained 40 mM sodium citrate, pH 5.5, 0.3 mM [7-³H]-testosterone, 1 mM dithiothreitol and 500 μM NADPH in a final volume of 100 μL. Typically, the assay was initiated by the addition of 50-100 μg prostatic homogenate or 75-200 μg scalp homogenate and incubated at 37° C. After 10-50 min the reaction was quenched by extraction with 250 μL of a mixture of 70% cyclohexane: 30% ethyl acetate containing 10 μg each DHT and T. The aqueous and organic layers were separated by centrifugation at 14,000 rpm in an Eppendorf microfuge. The organic layer was subjected to normal phase HPLC (10 cm Whatman Partisil 5 silica column equilibrated in 1 mL/min 70% cyclohexane: 30% ethyl acetate; retention times: DHT, 6.8-7.2 min; androstanediol, 7.6-8.0 min; T, 9.1-9.7 min). The HPLC system consisted of a Waters Model 680 Gradient System equipped with a Hitachi Model 655α Autosampler, Applied Biosystems Model 757 variable UV detector, and a Radiomatic Model A120 radioactivity analyzer. The conversion of T to DHT was monitored using the radioactivity flow detector by mixing the HPLC effluent with one volume of Flo Scint 1 (Radiomatic). Under the conditions described, the production of DHT was linear for at least 25 min. The only steroids observed with the human prostate and scalp preparations were T, DHT and androstanediol.

Inhibition Studies

Compounds were dissolved in 100% ethanol. The compound to be tested was pre-incubated with the enzyme (either 5α-reductase type 1 or 2) prior to initiation by addition of substrate testosterone. IC₅₀ values represent the concentration of inhibitor required to decrease enzyme conversion of testosterone to dihydrotestosterone by 50% of the control. IC₅₀ values were determined using a 6 point titration where the concentration of the inhibitor was varied from 0.1 to 1000 nM. Representative compounds of this invention were tested in the above described assay for 5α-reductase type 1 and type 2 inhibition.

A compound referred to herein as a 5α-reductase 2 inhibitor is a compound that shows inhibition of the 5α-reductase 2 isozyme in the above-described assay, having an IC₅₀ value of about or under 100 nM.

The compounds are tested in the above-described assay for 5α-reductase type 1 and type 2 inhibition, and were found to have IC₅₀ values under about 100 nM for inhibition of the type 1 isozyme. Compounds found to have IC₅₀ values of under about 50 nM for inhibition of the type 1 isozyme are called type 1 inhibitors.

The compounds called “dual inhibitors” were inhibitors of both 5α-reductase type 1 and 5α-reductase type 2 as defined above.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the subject being treated for any of the indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A method for determining the risk of a urologic event in a man, comprising measuring the serum PSA of the man.

2. The method according to claim 1, wherein the urologic event is selected from BPH-related surgery and acute urinary retention.

3. The method according to claim 2, wherein the urologic event is BPH-related surgery.

4. The method according to claim 2, wherein the urologic event is acute urinary retention.

5. The method according to claim 1, wherein the total serum PSA of the man is measured.

6. The method according to claim 1, wherein free serum PSA of the man is measured.

7. The method according to claim 1, wherein the percent free serum PSA of the man is measured.

8. The method according to claim 1, for determining the risk of a urologic event in a man, comprising measuring the serum PSA of the man, and determining if the value is over 1.3 ng/mL.

9. The method according to claim 1, for determining the risk of a urologic event in a man, comprising measuring the serum PSA of the man, and determining if the value is over 3.3 ng/mL.

10. A method for reducing the risk of a urologic event in a man at risk for a urologic event according to risk determination method of claim 1, comprising administration of an inhibitor of 5α-reductase to the man.

11. A method for reducing the risk of a urologic event in a man at risk for a urologic event according to risk determination method of claim 1, comprising administration of a compound of structural formula I to the subject: 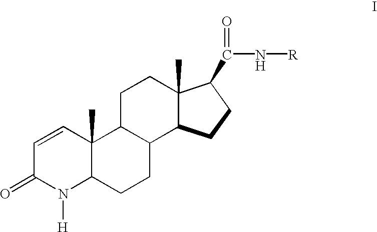
wherein R is selected from:
(a) C₁₋₁₀ alkyl, unsubstituted or substituted with one to three halogen substituents, and
(b) phenyl, unsubstituted or substituted with one to three substituents independently selected from halogen, methyl, and trifluoromethyl;
or a pharmaceutically acceptable solvate or crystal form thereof.

12. The method according to claim 11, wherein R is selected from:
(a) unsubstituted C₁₋₁₀ alkyl, and
(b) phenyl unsubstituted or substituted with one or trifluoromethyl substituents.

13. The method of claim 11, wherein R is t-butyl.

14. The method of reducing the risk of precipitated acute urinary retention according to claim 11, wherein R is 2,5-bis(trifluoromethyl)phenyl.

15. The method according to claim 9, comprising administration of finasteride at a dose of 5 mg per day.

16. A method for reducing the risk of a urologic event in a man at risk for a urologic event by having a serum PSA level of over 1.3 ng/mL, comprising administration of an inhibitor of 5α-reductase to the man.

17. The method according to claim 16, comprising administration of a compound of structural formula I to the subject: 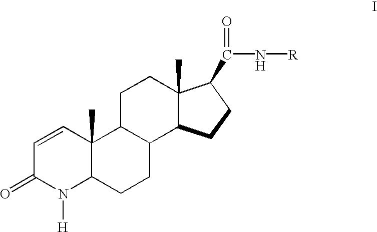
wherein R is selected from:
(a) C₁₋₁₀ alkyl, unsubstituted or substituted with one to three halogen substituents, and
(b) phenyl, unsubstituted or substituted with one to three substituents independently selected from halogen, methyl, and trifluoromethyl;
or a pharmaceutically acceptable solvate or crystal form thereof.

18. The according to claim 17, wherein R is selected from:
(a) unsubstituted C₁₋₁₀ alkyl, and
(b) phenyl unsubstituted or substituted with one or two trifluoromethyl substituents.

19. The method of claim 17, wherein R is t-butyl.

20. The method of reducing the risk of precipitated acute urinary retention according to claim 16, wherein R is 2,5-bis(trifluoromethyl)phenyl.

21. The method according to claim 16, comprising administration of finasteride at a dose of 5 mg per day.

22. A method for reducing the risk of a urologic event in a man at risk for a urologic event by having a serum PSA level of over 3.3 ng/mL, comprising administration of an inhibitor of 5α-reductase to the man.

23. The method according to claim 22, comprising administration of a compound of structural formula I to the subject: 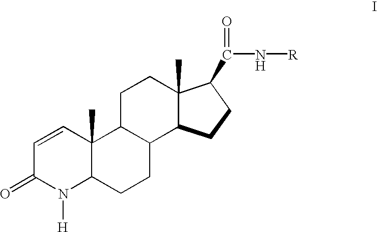
wherein R is selected from:
(a) C₁₋₁₀ alkyl, unsubstituted or substituted with one to three halogen substituents, and
(b) phenyl, unsubstituted or substituted with one to three substituents independently selected from halogen, methyl, and trifluoromethyl;
or a pharmaceutically acceptable solvate or crystal form thereof.

24. The method according to claim 22, comprising administration of finasteride at a dose of 5 mg per day.

25. A kit for determining the risk of a urologic event in a male subject comprising a test for serum PSA measurement and a means for relating the serum PSA measurement to the risk of a urologic event.
